(19) United States
(12) Patent Application Publication
(10) Pub. No.: US 2010/0145160 A1
(43) Pub. Date: Jun. 10, 2010

(54) MULTIMEDIA, MULTISERVICE AND CONNECTABLE MOBILE ASSEMBLY FOR DIAGNOSIS, PRESCRIPTIONS, MEDICAL CHECKUPS AND NURSING CARE

(76) Inventors: Jacques Cinqualbre, Rosheim (FR); Joseph Puzo, Montmirail (FR)

Correspondence Address: DAVIS & BUJOLD, P.L.L.C., 112 PLEASANT STREET, CONCORD, NH, 03301, US

(21) Appl. No.: 12/439,378

(22) PCT Filed: Aug. 30, 2007
(86) PCT No.: PCT/FR2007/001413

§ 371 Date: Nov. 13, 2009

(30) Foreign Application Priority Data
Aug. 30, 2006 (FR) 0607618

(51) Int. Cl. A61B 5/00

(52) U.S. Cl. 600/300

(57) ABSTRACT

Connectable, multimedia and multiservice mobile assembly for diagnosis and medical checkups, characterized in that it is composed of a directional rolling base (2) and of a cylindrical hollow body (3) enclosing electrical power supply means (28, 29), identification means (18), computing means and multimedia communication means as well as a facility (39) for disinfecting the hands and in that it is predisposed for its reception and connection to a docking station. This invention is of particular interest to manufacturers of trolleys intended for hospitals and care units.

This application is a National Stage completion of PCT/FR2007/001413 filed Aug. 30, 2007, which claims priority from French patent application serial no. 06/07618 filed Aug. 30, 2006.

### FIELD OF THE INVENTION

The present invention is related to a multi-media, multi-service, connectable mobile assembly serving as a professional office for the diagnosis, the prescriptions, the medical checkups and nursing care.

It also provides two additional functions: on one hand keeping track of orders, acts and prescriptions and on the other, hand, prevents, in part, infections transmitted between individuals.

### BACKGROUND OF THE INVENTION

In hospitals, nursing homes and other analogous sites, rounds for patients examination are made by physicians and nurses, together or separately.

Theoratically, rounds consist of an evaluation of the current clinical status of the patient, and correlating it to his past history and to complementary data gathered from lab profiles, imaging and consultant's opinions.

Practically, this collection of data is even now mostly performed by nurses and medical students who painfully gather information provided on paper. One can easily imagine the delays, the risk of errors and the redundancies which lead to loss of valuable time, especially with frequent shortage of care providers.

When the results are available through the hospital information system, they are delivered on fixed desktop stations away from the patient's room. This leads to back and forth transfers with multiple phases of writing of one's notes. This method is rather unsatisfactory and, on the whole, the hospital information system remains under-utilized.

Portability issues for medical information have lead to design a cart loaded with a true mobile station. Indeed the attempts of using PDAs have been showing quick limitations for managing the data with a low-quality display. Tablet PCs are favored by some supporters, notwithstanding the obligation to carry them, and to cope with discomfort due to their volume, risks of fall; and individual usage which is not compatible with the team approach for making rounds.

Most frequent experiences consist in placing a laptop on a cart without changing the unsatisfactory individual practice.

Moreover, even if all of these solutions more or less remedy the cited problem, none of them solves the second part of the rounds, namely to perform a computerized physician prescription.

Care providers, physicians as well as nurses, use their hands, not to carry or to use informatics, but to greet, examine, move the patients . . . and mostly to prescribe, drugs, cares, or tests or treatments and to write progress notes. Habits within the hospitals bear still largely on the handwriting message with cursive script.

The aim of the present invention is to improve the practice of making rounds, care and the follow-up of patients both within and outside the hospital by providing a mobile multi-media, multi-service station serving as a comprehensive information, services and care data base.

Thus, the invention is quite able to transfer the data captured by its integrator software.

This mobile station fully embodies a digital handwriting process instantly transformed in direct electronic capture with recognition and transposition into print characters and computable language. This important function has a direct impact on the physical characteristics and ergonomic features of the mobile cart according to the invention.

### SUMMARY OF THE INVENTION

The object of the present invention is to provide a global answer to this field of constraints by integrating in the same concept, the creation of a professional mobile office able to gather all the necessary medical and administrative data as a whole and the creation of a natural interface of instruction providers which fully integrates daily practice of providers, while suppressing re-writing uncertainties, errors and transmission failures.

The mobile unit according to the invention also provides an accurate tracking by identification of the manual script, and its author, time stamping which leads to identifying this unique document ending in this paper form which moreover represents the incontestable material proof frequently required from a hospital computer department. Furthermore, in case of system failure, the care process can be further carried out without major disturbances due to the fact that no computerized data will be lost since it will be reintegrated into the health information system, with actual time of capture, at the time the system function will be restored.

### BRIEF DESCRIPTION OF THE DRAWINGS

Other characteristics and advantages of the invention will appear in the following description, provided as an example with the appended drawings which are:

FIG. 1: a general perspective view of the mobile system according to the invention,

FIG. 2: a composite front and lateral view of the mobile system according to the invention,

FIG. 3: a composite sectional view of the cylindrical body and lateral aspects of the base and of the upper part of the mobile system according to the invention,

FIG. 4: a perspective schematic view of the housing holes located in the upper part,

FIG. 5: a perspective schematic view of housing cavities for the mouse and the bar-code reader,

FIG. 6: a lateral view of the multi-directional rolling platform of the mobile system according to the invention,

FIGS. 7 to 9: schematic plan views, showing the keyboard in hidden position then during extraction and final position outside of the drawer

### DETAILED DESCRIPTION OF THE PREFERRED EMBODIMENTS

The mobile unit according to the invention is direct at being used as a mobile diagnostic, prescribing and clinical follow-up station inside a hospital, a nursing home, a care unit or the like.

It provides additionally two further functions: on the one hand, tracking of material, prescriptions and acts; on the other hand prevention of hand-transmitted infections between individuals.

It is autonomous as to its mobility, its operation and the functioning of the devices that it carries due to an embedded independent electric power supply.

It is also self-communicating which means that it is capable of transferring and receiving messages and information with a network which is wirelessly linked to, e.g.; through a radioelectrical connection known as WiFi or other, directly or across a proxy base or station through which it is connected.

It can be plugged to a docking station to build a common gathering system receiving and processing information coming from different mobile units coming from different departments.

It can be plugged to a docking station for different purposes, especially for transferring data or information and recording work and configuration phases or any kind of procedures.

The mobile unit 1 according to the invention is generally composed of a multi-directional rolling platform 2, a hollow mainly cylindrical body 3 mounted on this platform 2 and at the upper part 4 of the cylindrical body 3, an inclined platform 5 constituting an inclined working plane 6 completed by a screen 7 mounted on an articulated stand 8, a digital writing pen 9 and a manual gripping element or shape 10 for pushing and guiding of the mobile unit for example shaped as a sphere which allows manual pushing, pulling and guiding of the same by a user.

The upper part 4 of the mobile unit 1 is closed by the inclined working plane 5 which is limited towards the bottom by a low linear stop for example of the stick type 11 to be used as a writing table 12 on a computer writing support 13 giving it the shape of a lectern. The upper part 4 also comprises the articulated stand 8 shaped to receive the screen 7, which may be a touch screen or not, as well as complementary devices such as the instrument of digital writing pen 9 for writing and capturing with automatic writing recognition processing by means of a suitable computer software.

The shape of the body 3 of the mobile unit 1 is cylindrical because this shape is non aggressive, self-protected against impacts and minimizes the number of sharp corners, protuberances and cavities which tend to host microorganisms, dust and dirt.

The hollow body 3 comprises in its inner part several housing apertures and places through its side wall which remain open or can be closed by a sliding, rotating or swivel flap.

Among these housing apertures the following should be noticed:

an open recess 14, for a control device known as a “mouse” 15 as well as a biometric sensor-identifier 16 which would recognize for example a digital print of the future user and after validation, allow starting of the mobile unit 1,

an open identification hole 17 consisiting of a base 19 and a mobile gripping handle 20 for identifying, by coded support, coded labels or else comprising a manual reader 18 for cards and codes, more specifically code-bars,

a flat open hole 21 with a slit-shaped entry 22 for a keyboard 23 placed on a extractible tablet 24 which is slideably mounted on a stand with sliding rails 25 which is itself pivotable and pulled by a gripping tab 26 to facilitate a large array of movements,

an obturable lower technical compartment 27 arranged in the lower part, housing an independent power supply or a so called auto-electric supply consisting of a rechargeable battery 28, a charge unit 29, a tension regulator and different organs and devices. This technical compartment 27 can be divided into several sub-units by walls of different heights. It is located below a middle compartment which can be divided into different levels by one or more horizontal panels as, for instance, 30 and 31 forming supporting planes at a higher level for different peripherals such as a printer 32 or a scanner 33 or other.

This compartment, at the middle level, also comprises in its upper portion a computer central unit 34 with linked to or integrated or built in different peripherals among which a reader 35 of different displaceable memory supports: cards, USB keys, mini-Cds and others placed on the panel 31 for instance.

The lower technical compartment is closed by a flapping door 36, with or without an openwork design.

The mobile unit comprises in the upper part a hand disinfection compartment 37 closed by a swivel flap 38 when it is not used, and arranged for a disinfection unit 39. The disinfection unit is housed in a technical compartment or enclosure 40 isolated from the internal space of the mobile unit and preferably separated from the outside by means of a flexible panel 41 having an opening 42 for access of the hands. It is a disinfection unit for the hands, for example by blowing or spraying. This unit comprises a plurality of blowing or spraying nozzles or orifices such as 43 oriented preferably towards the median part of the enclosure corresponding to a volume 44 being left free for positioning the hands above a receptacle 45. Each one of these nozzles or orifices 43 blows or directs on the hands a gas flow of a biocompatible disinfection product, which evaporates, or a mist of a biocompatible disinfection fluid which preferably evaporates easily.

This disinfection compartment is favorably arranged at the hand level of a medium sized individual, for instance in a compartment placed below the working plane 5.

The hollow body 3 of the mobile unit 1 comprises in the technical compartment 27, which is closed by a sliding or flapping door 36, with or without an openwork design, a place for the battery 28 for instance made of gel and its specific charging unit 29 and different other places for ancillary devices devoted to power supply and to the general operation of the mobile unit 1.

Other compartments are arranged in the middle level. Non limitingly, these are the following :

a place for the central computer unit 34 and its multi-support reader 35

a place for a wireless communication device 46, for instance of WiFi type

Very careful attention has been given to a maximal aeration and if necessary ventilation of the inner space of the technical compartment 27 which may be exposed to dangerous high temperatures and this by a ventilation device if necessary. A signal diode lamp indicating the charge battery status is placed at this level or optionally at any other more accessible place of the mobile unit 1.

As other embodiments, one can imagine several computer central units of different kinds. In a common form, it is a micro-computer, compact, thoroughly attached and equipped with all suitable desired connectivity including platforms for additional ports (USB, Fire Wire . . . ). Cards or communicating keys can be inserted at this level. There, through an adequate window, can be inserted a printer 32 with a one to one feeding for isolated sheets such as prescriptions edited at the bedside and printed immediately.

As previously described, the upper part comprises, as a standard equipment, the three following compartments accessible through open recesses, as well as the compartment for the disinfection unit 39.

The first one, 21, in the form of a transversal slit 22, serves to house the retractable keyboard 23 with numeric keypad placed on a panel 24 which can be mobilized through a procedure described in the series of FIGS. 7 to 9. The tab 26 enhances the gripping to pull it out of its compartment.

To its left, aside, (although a left-right substitution is possible as well as an embodiment allowing to place, regardless their orientation, the keyboard and the mouse, as wished through modules which fit into the mobile unit), an open recess 14 houses a mouse 15, and its entry opening allows the user's hand to pass in order to perform blind mobilization of the mouse. Of course, the floor of this compartment is covered with a mouse pad or a similar covering.

On the left vertical flank of the open recess 14 of the mouse compartment, the biometric sensor-identifier 16 has been placed to give the right of access to the information system.

Below the open recess 14 of the mouse compartment 15, an other compartment, 17, identical or similar for aesthetically compatible reasons, will host the bar-code reader 18, for example of the wireless type.

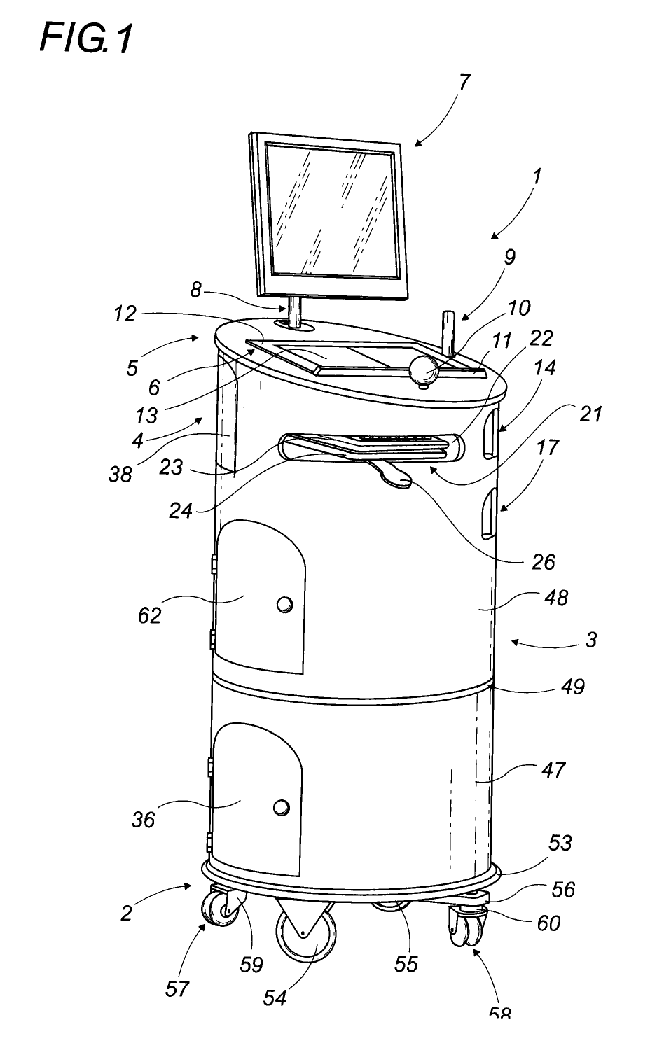

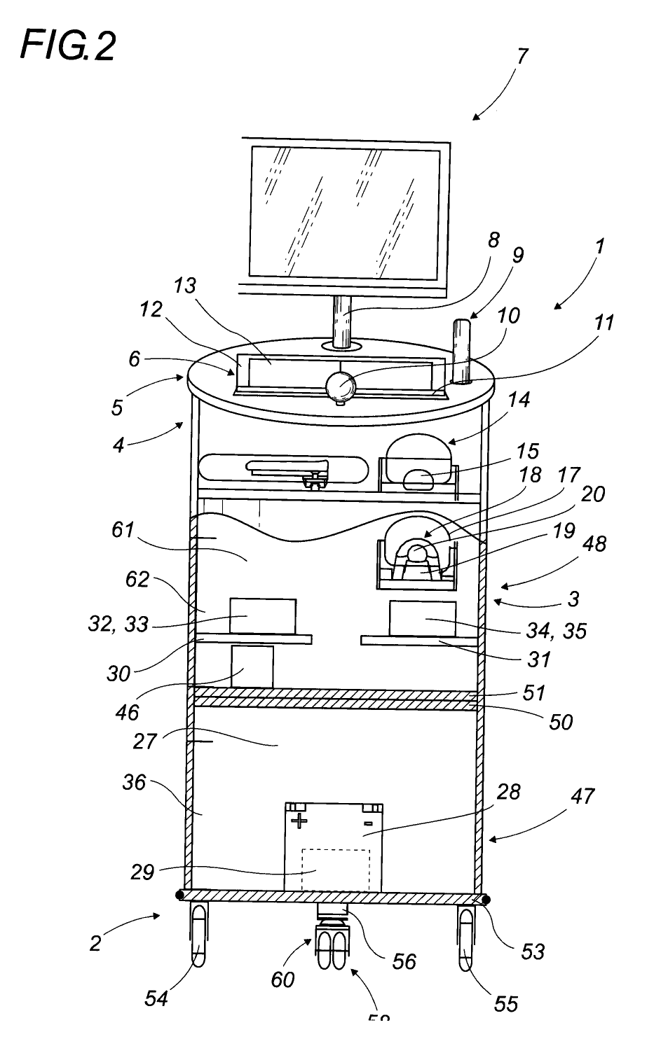

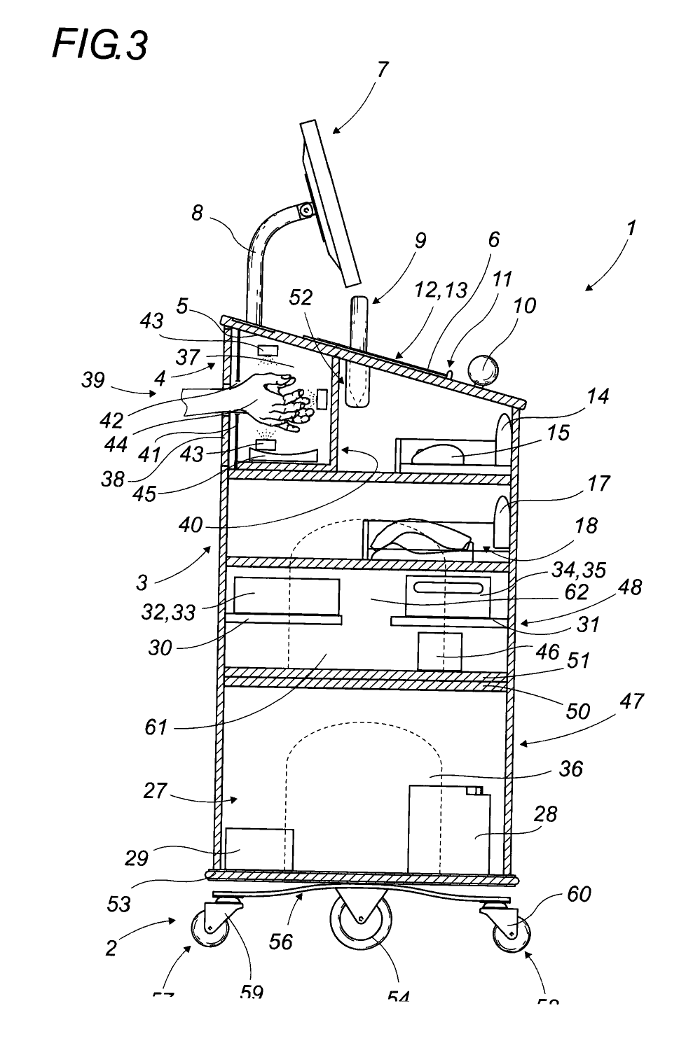

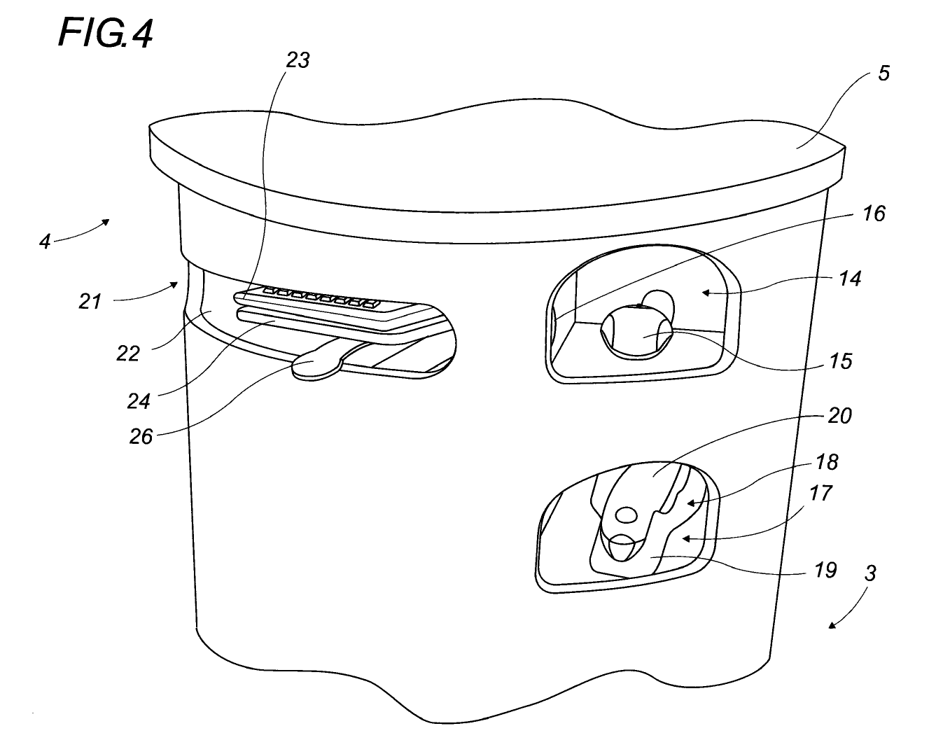

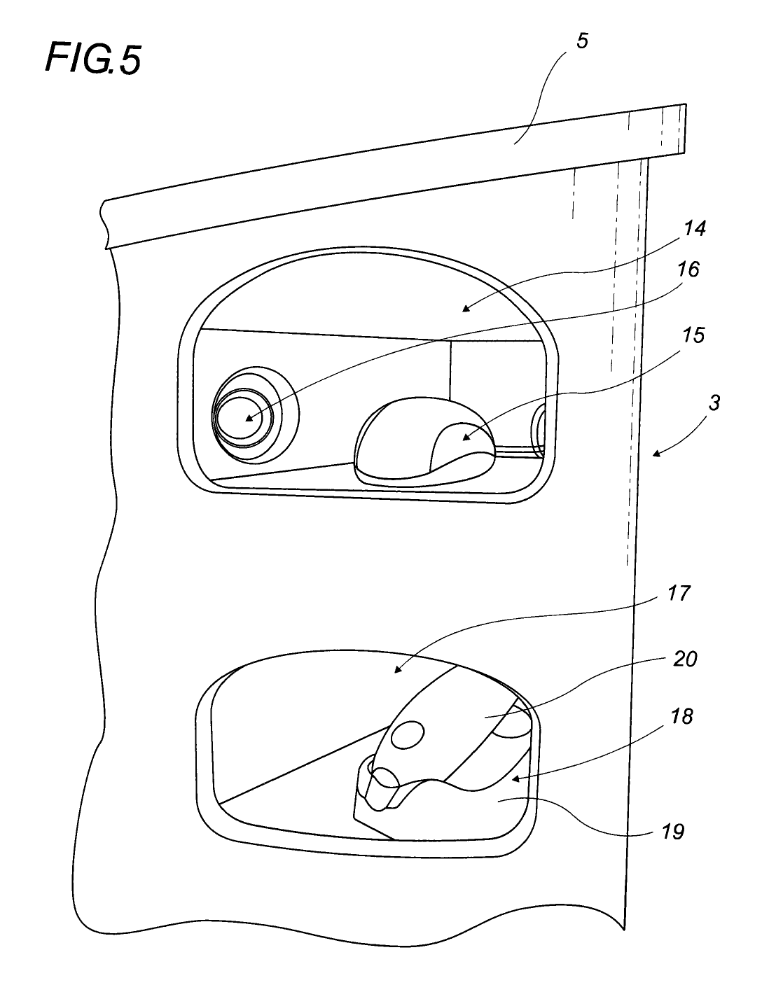

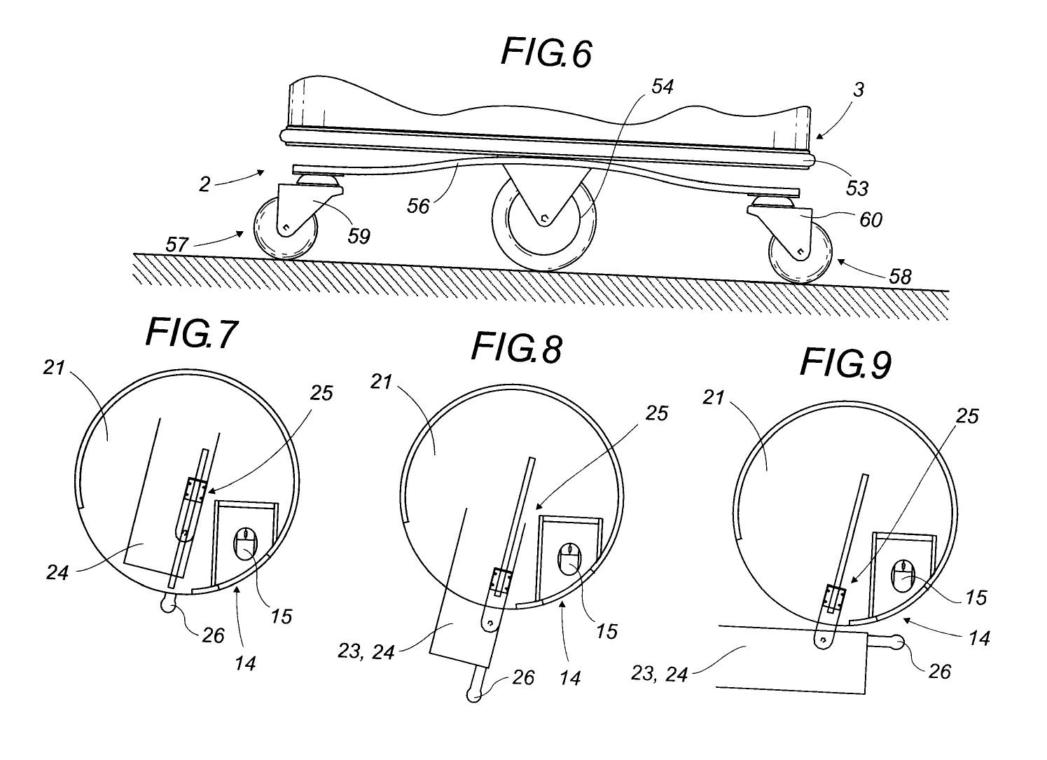

The cylindrical body 3 of the mobile unit 1 can be composed of two parts 47 and 48, separated by a double line 49 on the FIG. 1 corresponding to the two panels, 50 and 51 shown on the sectional views. Thus, it is possible to modify, by shortening or extending, part of the global height, which results in facilitating the industrialization process of some smaller models.

Globally, the upper part 4, which is the working surface 5, terminates the cylindrical body 3 which is the extremity of the slantingly cut hollow body 3 beyond which it extends in an even way, resulting in giving it an elliptical form. Its obliquity and general form and its writing and reading function provide the appearance of a lectern and its use as such. This working surface 5 only consists in a writing table 12 limited at the bottom by a transversal linear stop, for example a stick 11 placed immediately above the guiding sphere 10 in order to hold the paper forms placed on it. A specific compartment 52 is devoted to the writing instrument 9 of the digital writing pen type. This pen is used to write on specific type paper which transfers all captured data to the information system with an identification and horodating of the document. This compartment 52 is a pen holder and is a receiving compartment with an axis parallel to the one of the body 3 of the mobile unit 1, which maintains the pen in erect position. It is fixed in a non visible manner below the working table.

At the forefront of the working table is fixed the articulated stand 8 of the screen 7, either of the touch screen type or not, which can be tilted antero-posteriously.

On the vertical line of the uppermost part of the mobile unit, close to the screen's stand, a through hole is located at mid-height and facing the computer compartment, which allows to pull out the electric cable for the battery charger as well as the cable to connect the computer to a video-projector.

The dimensions of the body 3 of the mobile unit 1 can be modified according to the needs but several rules must be respected: the dimension of the upper table should be able to receive a paper sheet of the A3 (European) legal size and its height should be over any individual whose size is between 1.6 and 1.9 meter.

The rolling base 2 of the mobile unit 1, according to the invention, bears the body 3 through a carrying plate 53 on which it is mounted.

This carrying plate 53 serves as an interface to a rolling, steerable and multidirectional base.

This carrying plate 53 serves as a frame or is secured to an intermediate support which serves as a frame.

On the frame or directly on the additional plate 53 are placed two free rotating wheels or casters diametrically opposed and fixed in their direction.

At the center of the frame or of the disk 53 is placed on its middle part a fixed or mobile arm 56 affecting the general shape of on arch carried on each extremity by a caster, or a pair of two directional casters such as 57 and 58, fully free both in rotation and in direction.

They allow, with a strictly median push, a linear progression without the need to correct rotations or translations.

Each caster 57 and 58, or pair of casters, is mounted pivotally free by means of a common device easily available on the market, for example a pivoting support 59 or 60 one of the ball bearing type or analog.

The arch shape of this directional arm 56 allows easy passage over small obstacles or deformations of the running plan.

The directional arm 56 is preferably flexible. For this purpose it is made of a suitable flexible matter or material, either metallic or lamellated-sticked wood or a composite material.

Wheels or casters fixed in direction 54 and 55 can be motorized, for example by using an existing easily available motor or devoted motor-wheels, in order to facilitate the mobility in maneuvers and movements of the mobile system.

Of course, in case of a pivoting directional arm, the motor can be inserted in the center at the pivoting point in order to assist or pilot the system from a distance.

The compartment 61 at the middle level is closed by a flapping or sliding door 62 to give access to the inner volume in which the computer is housed.

The mobile unit 1 is completed with the manual thrust means and guiding device 10 which is an element or a conformation for gripping, pushing or steering, for instance in the form of a recess in the upper platform or of a handle, either solid or hollowed, mounted in an upright position at the rear part or the body of the mobile unit. It can also affect, as shown on the attached figures, the form of a sphere either lightly raised over the working platform 5 of the mobile unit 1, or directly secured to it.

The rolling base can be provided with a parking or service brake or a driving brake.

In the manual operation mode, the user pushes and guides the mobile unit 1 by using this pushing and steering handle 10 with one single hand.

When moving the mobile unit 1 according to the invention, stability is insured by seating on the four bearing points acting as pressure points carried out by the casters, in such a way that changing direction and passing over obstacles and deformations is quite easy by manual pressure on the pushing and steering handle combined with the flexibility of the directional arm. This brings further a suspension effect allowing to soften the shocks and damper the jerks during the maneuvers and the passing over.

The result of this inventive device is exceptional mobility which leads to the absence of a conventional steering wheel or device, replaced by the sphere 10 placed on the upper part 4 of the mobile unit 1, and sufficient for precise steering the mobile unit. This system of pushing-steering could, in a motorized version of the mobile unit 1, be handled in order to trigger the starting of the electric motor-wheels placed on, or in place of, the two fixed in direction casters 54 and 55.

Several supplementary and optional equipment are possible to increase performances and services rendered according to the specificity of some applications.

According to the selected system of videoconferencing, speakers can be placed either laterally on the flanks or be flush with the upper platform. The microphone, if not included in the camera, can be placed at different levels.

A digital clock or a clock with hands, with or without chronometer function, can be inserted in a highly visible place.

Optionally, another open compartment can be placed in order to house one or several apparatus for measuring parameters, either with digital output and therefor with direct entry into the data processing system, or with conventional operation.

A plurality of buttons, the specific function of which is to be defined (selected call, emergency call, specific order . . . ), switching from an application to another, image display and reading, videoconferencing can be integrated either on a vertical panel placed in front of the user or on the working plan.

A digital camera will be arranged in a suitable compartment.

As well, a digital dictation system can be arranged.

It is the same for a geo-localization system using an RFID chip.

One will recall that the mobile system can be motorized by means of electrical motors, of the motor-wheel type, with adapted batteries for this use.

Finally, the disinfection station is placed either laterally at the upper part or, once more, on the longest slope line close to the foot of the screen.

Several variations will be described thereafter.

There is a version with shortened body in which the number of devices and equipment of which is reduced.

This embodiment could be used as a bedside table in a light version.

Other examples of light versions are listed below:

hospital model version with logistics functions such as nutrition restrains or psychological sustains

home care model version with different domestic functionalities such as: controls of apparatus, anti-breach safety, television function, interactive terminal . . . .

nursing home model version including imaging and therapeutic tools or devices or instruments for measurement such as : audiometry, optometry . . . .

educational model version to serve as a robot for presenting didactic tests.

The mobile unit, according to the invention, can be adapted for specific uses in medical or surgical sites such as operating rooms, technical (cath-labs, endoscopies . . . ) emergency rooms, recovery rooms and intensive care units. In these cases, if required, the mobile unit can be resistant to electromagnetic radiations and their interferences.

The mobile unit can be used in cooperation with a docking station designed for instance as a desk, a flank of which comprises a receiving lodging for plugging the mobile unit to its receiving means.

The mobile unit according to the invention is arranged to be connected to the docking station in order to communicate, to discharge and to load information.

To keep a sufficient maneuverability and essential electrical autonomy, the mobile unit has to be provided with a limited number of ancillary equipment in order to avoid overload and not to compromise the reliability of the embarked electronic devices.

This choice leads, in a given configuration, to limit the possibilities, on the one hand, to capturing data resulting in the patient electronic record, and, on the other hand, emitting specific instructions as to cares, therapeutic conclusions and requests for complementary examinations and/or specific medical advice. A common micro-computer is enough to carry out this requirement, with a bar-code reader and a mini printer to edit, at the bedside, a discharge prescription without seeking to overpass this limited objective. A built-in manual disinfection unit is present on the basic version.

All other clerical functions will be devoted to a docking base or station with an adapted configuration including a specific area to host the mobile unit and all technical plugs necessary for its connection. For reasons of clarity and complete description, the functions of the mobile unit according to the invention are recalled thereafter. Those functions are as follows:

Reading of all data related to a given patient from the hospital information system (SIH) or from the patient's personal health record (DMP).

Computerized physician order entry and scheduling.

Day by day medical and nursing record's implementation.

Tracing and documenting all acts, prescriptions, disposable products and materials with recording and execution of medical acts.

Contacts with the continuum of care providers of the related patient inside and outside the institution.

Completing the patient's and care unit's databases for possible data mining, for scientific, medical, public health or economical purposes through connection with appropriated software and databases.

Prevention of manually transmitted nosocomial infections by care providers.

Functions of providing light of and from the mobile unit, of storage and tidying.

Reading the data of imaging display or biology.

Therapeutic direct function through embarked tool (defibrillator).

A rapid description of the main components follows also.

Directional rolling base with optional brakes to be handled with one hand.

Independent electrical supply with a sufficient autonomy (12 hours autonomy minimum) with full compliance with sanitary rules and flexibility for 12-20 volts to 220 volts range. Use of specific charger, or optional induction charger.

Electrical power supply platforms and hubs for USB, FireWire . . . .

Connected printer, inside or remote.

Connected scanner, inside or remote.

Central computer unit(s) comprising micro-computers or a combination thereof (one up to three central units).

IP phone on WiFi.

Wireless bar-code reader.

Personal biometric features reader.

RFID positioning reader.

Digital voice recording with voice recognition.

Autonomous or integrated microphone with sound/image shift correction.

Incorporated speakers.

Active or passive steering and guiding sphere, controlling or not the starting of the motor wheels.

Hidden retractable compact keyboard.

Hidden mouse in designated recess.

Inclined working handwriting panel with horizontal stick at the bottom.

Screen with or without touch-function, eventually integrating camera and speakers.

Digital camera/video recorder with internet connection.

Digital pen and intercommunication paper.

LEDs to light a part of the mobile unit or for surroundings lighting purposes.

Digital clock.

Electrical motorization of the mobile unit (motorized rollers of the motor-wheel type) with adapted battery.

Distance remote control and guiding of the mobile unit.

Built-in disinfection unit.

Imaging and assessment devices.

Ultrasound imaging systems.

Cardio-respiratory therapeutic urgency tools of the defibrillator type, for example semi-automatic defibrillator (SDA)

1-21. (canceled)

22. A mobile multimedia and multi-service mobile unit for diagnosis and medical monitoring, the mobile unit comprising:
a directional rolling base and a hollow body comprising means of power supply,
means of identification,
a micro-computer,
means for data processing and multimedia means of communication, and
a hand disinfection station for disinfecting hands without water formed in a cavity of the body of the mobile unit, and
an inclined planar upper face for at least one of hand writing and keyboard writing, the mobile unit being a working platform and with various compartments which house a control means for and peripherals of the micro-computer, and
the mobile unit being predisposed for reception and connection at a docking station.

23. The mobile unit according to claim 22, wherein the working platform has an area for writing on a paper medium, and the writing area is delimited at a lower end by a linear stop.

24. The mobile unit according to claim 22, wherein the working platform comprises a display screen, and a base of which is located at an upper front part of the inclined plane.

25. The mobile unit according to claim 22, wherein the working platform comprises a compartment for a writing instrument.

26. The mobile unit according to claim 25, wherein the writing instrument is a digital pen.

27. The mobile unit according to claim 22, wherein the cylindrical body includes several stages with a plurality of compartments.

28. The mobile unit according to claim 27, wherein one of the compartments houses a micro-computer input device.

29. The mobile unit according to claim 27, wherein one of the compartments houses a biometric identification means.

30. The mobile unit according to claim 27, wherein a lower stage is a multi-compartment and multi-site stage which houses a battery and a charger.

31. The mobile unit according to claim 22, wherein a housing (37) is closed by a swivel flap (38) and contains the hand disinfection unit (39) in a technical enclosure (40) that is isolated from an interior volume of the mobile unit and separated from an exterior of the mobile unit by a flexible panel (41) with an opening (42) for access of the hands.

32. The mobile unit according to claim 31, wherein the hand disinfection unit (39) disinfects hands by one of blowing or spraying and comprises a plurality of either blowing or spraying nozzles (43) that are directed toward a median part of the enclosure which is open and receives the hands above a receptacle (45), each of the nozzles(43), either blows or sprays a gas flow on the hands, the gas flow being either a bio-compatible disinfection product which evaporates or a mist of a biocompatible disinfection fluid.

33. The mobile unit according to claim 22, wherein the steerable and multi-directional rolling base (2) is located in a lower part of the mobile unit, under a carrying plate (53), and comprises an interface, two casters fixed in direction, a directional arm assembled in a center of the carrying plate and two free rotating casters, with one caster is located at each end of the directional arm (56).

34. The mobile unit according to claim 33, wherein the directional arm (56) is fixed.

35. The mobile unit according to claim 34, wherein the directional arm (56) is pivotable.

36. The mobile unit according to claim 34, wherein the directional arm (56) is fixed and flexible.

37. The mobile unit according to claim 33, wherein the directional arm (56) is in a general shape of an arch.

38. The mobile unit according to claim 33, wherein the carrying plate (53) is a frame.

39. The mobile unit according to claim 33, wherein the carrying plate (53) is supplemented by a frame.

40. The mobile unit according to claim 33, wherein the casters fixed in direction and one of motorized and motorized wheels.

41. The mobile unit according to claim 22, wherein the mobile unit comprises means adapted for receiving the mobile unit in a housing and connection to a docking station for transfers of data and information and recording and configuration work.
